(19) United States
(12) Patent Application Publication
(10) Pub. No.: US 2005/0025744 A1
(43) Pub. Date: Feb. 3, 2005

(54) Combination therapies for multiple sclerosis

(76) Inventor: Thomas E. Lane, Irvine, CA (US)

Correspondence Address: O'MELVENY & MEYERS, 114 PACIFICA, SUITE 100, IRVINE, CA, 92618, US

(21) Appl. No.: 10/903,118

(22) Filed: Jul. 29, 2004

Related U.S. Application Data
(60) Provisional application No. 60/491,735, filed on Aug. 1, 2003.

(51) Int. Cl. A61K 39/395; A61K 38/21

(52) U.S. Cl. 424/85.6; 424/145.1; 514/2

(57) ABSTRACT

The invention discloses methods and compositions for treating Multiple Sclerosis through the administration of humanized anti-IP-10 antibody in combination with an additional therapeutic compound.

### RELATED APPLICATION INFORMATION

This application claims the benefit of U.S. Provisional Application No. 60/491,735, filed Aug. 1, 2003.

### FIELD OF THE INVENTION

The present invention relates generally to methods for treating demyelinating disorders, particularly Multiple Sclerosis, with combination therapy. Specifically, the invention relates to methods for treating chronic progression of Multiple Sclerosis, as well as reducing the severity of Multiple Sclerosis symptoms, using combination therapy comprising therapeutically effective amounts of humanized anti-IP-10 antibodies together with another MS therapeutic compound.

### BACKGROUND OF THE INVENTION

Multiple Sclerosis (MS) is a T-cell dependent, demyelinating disease of the central nervous system (CNS) and spinal cord. In the course of the disease, scleroses i.e., lesions or scars, appear in the myelin sheath of nerve cells, disrupting transmission of electrical signals. Scleroses accumulate over time and result in the debilitating symptoms experienced by MS patients. MS is believed to be an autoimmune disease.

MS patients generally experience one of four clinical courses of disease, each of which might be mild, moderate, or severe: relapsing-remitting, primary progressive, secondary progressive, or primary-relapsing. About 85% of MS patients have the relapsing remitting form of the disease, in which they experience clearly defined relapses (also called flare-ups or exacerbations), which are episodes of acute worsening of neurologic function, followed by partial or complete recovery periods (remissions) that are free of disease progression.

Patient management focuses on wellness and therapeutic intervention. Therapeutic intervention is currently available with a variety of approved treatments falling into three main categories: 1) Treatment for the underlying course of the disease, i.e., chronic progression of MS; 2) Treatment of exacerbations; and 3) Treatment of specific MS symptoms. Treatment for the chronic progression of MS has been substantially advanced by the availability of the immunomodulator compounds beta interferon-1a (Avonex® (Biogen); Rebif® (Serono)); beta interferon-1b (Betaseron® (Berlex)); and glatiramer acetate (Copaxone®, Teva/Aventis). A number of positive outcomes have been demonstrated in people with relapsing-remitting disease; such positive outcomes include, for example, reduction in the frequency and severity of relapses, reduction of brain lesion development, as evidenced by magnetic resonance imaging (MRI), and the possibility of reduction of future disability. The immunosuppressor mitoxantrone (Novantrone® (Amgen, Serono) has also shown some efficacy for treating the chronic progression of MS.

The treatment choice for exacerbations is generally high doses of corticosteroids, usually methylprednisolone (Solu-Medrol, generic); treatment of specific symptoms are managed on a case-by-case basis and may employ administration of a variety of therapeutic compounds depending on the particular symptom.

While advances in therapeutic compounds and therapeutic strategies have greatly enhanced the lives of MS patients, the present invention sets forth new and improved methods for treating MS, and demyelinating diseases in general, with the object of further improving patient care.

### SUMMARY OF INVENTION

The present invention provides a method for treating chronic progression of MS in an individual by administering to the individual a therapeutically effective amount of humanized anti-IP-10 antibody in combination with a therapeutically effective amount of an immunomodulator, such as beta-interferon 1a, beta-interferon 1b, glatiramer acetate, and the like, or humanized anti-VLA-4-antibody. The present invention also provides a method for reducing the severity of MS symptoms in an individual by administering to the individual a therapeutically effective amount of humanized anti-IP-10 antibody in combination with a therapeutic compound useful for mitigating MS symptoms. Administrating the combination therapy of the present invention likely synergistically enhances the effect of treatment either with humanized anti-IP-10 antibody or the therapeutic compound when administered separately. Also provided is a kit containing humanized anti-IP-10 antibody packaged alone or together with a candidate combination therapeutic.

### DETAILED DISCLOSURE OF THE INVENTION

The present invention provides improved methods for treating MS in humans by administering humanized anti-IP-10 antibody in combination with one or more other therapeutic compounds used in treatment of MS.

Two distinct combination therapies are envisaged by the present invention. In the first combination therapy, which is useful for treating chronic progression of MS, humanized anti-IP-10 antibody is used in combination with immunomodulators, such as, for example, interferons or glatiramer acetate, but not in combination with immunosuppressors. Chronic progression combination therapy comprising humanized anti-IP-10 antibody, in combination with immunomodulators, targets the release of the IP-10 chemokine during the remission or baseline phase of MS. As used herein, “chronic progression” refers to the remitting phase of relapsing-remitting MS, as well as the secondary progressive and primary-relapsing forms of MS.

In the second combination therapy, which is useful for reducing severity of MS symptoms, humanized anti-IP-10 antibody is used in combination with symptom-relieving compounds such as, for example, muscle relaxants, antispastics, or potassium channel blockers. Symptom-relieving combination therapy targets release of the IP-10 chemokine, thereby reducing ambient levels in the vicinity of neurons to reduce T-cell recruitment and inflammation.

As used herein, “anti-IP-10 antibody” encompasses antibodies and antigen binding fragments thereof which have specific binding affinity for the chemokine IP-10. This definition includes but is not limited to antigen binding fragments including Fab, F(ab′)₂, Fc, Fd and Fv fragments and the like, non-naturally occurring antibodies, including, for example, single chain antibodies, chimeric antibodies, bifunctional antibodies, complementarity determining region-grafted (CDR-grafted) antibodies and humanized antibodies, as well as fully human antibodies. Further, the definition includes but is not limited to both polyclonal, monoclonal, humanized, or primatized antibodies against IP-10. Other aspects of the definition of “anti-IP-10 antibody” are described elsewhere below.

A description of the role and effect of anti-IP-10 antibody was first set out in PCT publication WO 0215932 and WO 0306045, incorporated herein by reference. The chemokine IP-10, described at length therein, is also known as CXCL10, interferon inducible protein of 10 kDa, Small inducible cytokine B10 [Precursor], Interferon-gamma induced protein; Gamma-IP10, and is the product of the gene alternately known as CXCL10 or SCYB10 or INP10. Those publications disclose the use of polyclonal anti-IP-10 antibodies to treat demyelinating disease, such as Multiple Sclerosis, and for inducing remyelination, for example after CNS injury.

Neutralization of IP-10 represents a significant treatment option for MS and CNS injury because it decreases the extent of T-cell infiltration that is responsible for neurological degeneration. Further details on the role of other chemokines in disease processes, and the importance of modulating chemokine networks in the treatment of various diseases are reviewed in Schwarz, MK and Wells, TNC. 2002. Nature Reviews Drug Discovery. 1:347-358.

As used herein, the term “treating chronic progression” refers to a decrease in severity of MS symptoms, an increase in frequency and duration of MS symptom-free periods, or a prevention of impairment or disability due to MS affliction. Clinical tests for measuring the efficacy of combination therapies for treating chronic progression measure the deterioration of physical symptoms associated with MS including, for example, relapse rate; vision loss; sensory loss; gait disorders, such as axial instability, hyperreflexia, loss of dexterity, and spasticity; bladder dysfunction; depression; and cognitive impairment. Other clinical tests for measuring the efficacy of combination therapies for “treating chronic progression” include the expanded disability status scale (“EDSS”), the MS functional composite (“MSFC”), as well as MTR, NAA/Cr. Biochemical tests for measuring the efficacy of combination therapies for treating chronic progression of MS may measure levels of myelin, integrity of the blood-brain barrier, perivascular infiltration of mononuclear cells, immunologic abnormalities, gliotic scar formation and astrocyte proliferation, metalloproteinase production, and impaired conduction velocity. Accordingly, any clinical or biochemical assay that monitors any of the foregoing may be used to determine whether a particular treatment is efficacious for treating chronic progression of MS in an individual following treatment of the individual with the methods of the present invention.

As used herein, the term “reduction in severity” refers to an arrest, decrease, or reversal in signs, physiological indicators, biochemical markers or metabolic indicators associated with MS. Symptoms of MS include, for example, neurological impairment and neuroinflammation. Physiological indicators of MS include, for example, demyelination of nerve fibers. Biochemical markers indicative of MS include, for example, myelin and gamma globulin. Demyelination and remyelination of nerve fibers may be detected by clinical methods known to those of skill in the art. For example, evoked potentials may be used to measure the speed with which nerve impulses travel along nerve fibers throughout the nervous system. Additionally, computer-assisted tomography (CT) may be used to scan the nervous system to detect nerve fiber demyelination or remyelination. Magnetic resonance imaging (MRI) also may be used to scan the nervous system to detect nerve fiber demyelination or remyelination without the use of x-rays. Such MRI-based tests for measuring the efficacy of combination therapies for treating chronic progression, measure, for example, enhancing lesions, T1 black holes and T2 lesion load. Lumbar punctures or spinal taps may be used to withdraw cerebrospinal fluid, which subsequently may be tested for levels of biochemical markers, such as, for example, myelin or gamma globulin. Accordingly, any of the foregoing may be used to detect whether the severity of MS symptoms has been reduced in a patient following treatment of the individual using the methods of the present invention. Certain of the tests for monitoring the efficacy of treating chronic progression of MS may be used interchangeably with tests for monitoring the efficacy of reducing the severity of MS symptoms as is known to those of skill in the art.

Preparation of Anti-IP-10 Antibody

Methods of preparing and isolating antibodies, including polyclonal and monoclonal antibodies, using peptide immunogens, are well known to those skilled in the art and are described, for example, in Harlow and Lane, Antibodies: A Laboratory Manual, Cold Spring Harbor Laboratory Press (1988). Non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies are well known to those skilled in the art (Hoogenboom et al., U.S. Pat. No. 5,564,332, issued Oct. 15, 1996; Winter and Harris, Immunol. Today 14:243-246 (1993); Ward et al., Nature 341:544-546 (1989); Harlow and Lane, Antibodies. A laboratory manual (Cold Spring Harbor Laboratory Press, 1988); Hilyard et al., Protein Engineering: A Practical Approach (IRL Press 1992); Borrabeck, Antibody Engineering, 2d ed. (Oxford University Press 1995); each of which is incorporated herein by reference).

An IP-10 specific antibody can be raised using as an immunogen a substantially purified IP-10 protein, which can be prepared from natural sources or produced recombinantly. Additionally, a peptide portion of an IP-10 protein, including synthetic peptides, may be used as an immunogen for an IP-10 specific antibody. Moreover, a non-immunogenic peptide portion of a IP-10 protein may be made immunogenic by coupling the hapten to a carrier molecule, such as bovine serum albumin or keyhole limpet hemocyanin, or by expressing the peptide portion as a fusion protein. Various other carrier molecules and methods for coupling a hapten to a carrier molecule are well known in the art (see Harlow and Lane, supra, 1988; see, also, Hermanson, Bioconjugate Techniques, Academic Press, 1996, which is incorporated herein by reference).

An anti-IP-10 antibody can be labelled so as to be detectable, or for therapeutic purposes, using methods well known in the art (Hermanson, supra, 1996; Harlow and Lane, supra, 1988; 5 chap. 9). For example, an IP-10 antibody can be linked to a radioisotope, fluorophore, or other detectable agent by methods well known in the art.

Methods for raising polyclonal antibodies, for example, in a rabbit, goat, mouse or other mammal, are well known in the art (Harlow and Lane, supra, 1988). The production of anti-peptide antibodies commonly involves the use of host animals such as rabbits, mice, guinea pigs, or rats. If a large amount of serum is needed, larger animals such as sheep, goats, horses, pigs, or donkeys can be used. Animals are usually chosen based on the amount of antiserum required and suitable animals include rabbits, mice, rats, guinea pigs, and hamsters. These animals yield a range of 1-25 mL per single bleed (Harlow and Lane, infra, 1988). Rabbits are very useful for the production of polyclonal antisera, since they can be safely and repeatedly bled and produce high volumes of antiserum.

Two injections two to four weeks apart with 15-50 pg of antigen in a suitable adjuvant such as, for example, Freund's Complete Adjuvant can be followed by blood collection and analysis of the antiserum.

In addition, monoclonal antibodies can be obtained using methods that are well known and routine in the art (Harlow and Lane, supra, 1988). A peptide portion of a protein such as IP-10 for use as an immunogen can be determined by methods well known in the art. Spleen cells from an IP-10 immunized mouse can be fused to an appropriate myeloma cell line to produce hybridoma cells. Cloned hybridoma cell lines can be screened using a labeled IP-10 protein to identify clones that secrete anti-IP-10 antibodies. Hybridomas expressing anti-IP10 monoclonal antibodies having a desirable specificity and affinity can be isolated and utilized as a continuous source.

Humanized antibodies can be constructed by conferring essentially any antigen binding specificity onto a human antibody framework. Methods of constructing humanized antibodies are useful to prepare an antibody appropriate for practicing the methods of the invention and avoiding host immune responses against the antibody when used therapeutically. Current leaders in this field are Medarex, Inc. (Princeton, N.J.), Abgenix, Inc. (Freemont, Calif.) and Protein Design Labs, Inc (Fremont, Calif.).

The antibody described above can be used to generate human therapeutic agents by methods well known in the art such as complementary determining region (CDR)-grafting and optimization of framework and CDR residues. For example, humanization of an antibody can be accomplished by CDR-grafting as described in Fiorentini at al., Immunotechnology 3(I): 45-59 (1997), which is incorporated herein by reference. Briefly, CDR-grafting involves recombinantly splicing CDRs from a non-human antibody into a human framework region to confer binding activity onto the resultant grafted antibody, or variable region binding fragment thereof.

Once the CDR-grafted antibody, or variable region binding fragment is made, binding affinity comparable to the non-human antibody can be reacquired by subsequent rounds of affinity maturation strategies known in the art. Humanization of antibody in the form of rabbit polyclonal antibodies can be accomplished by similar methods as described in Rader et al., J. Biol. Chem. 275(18): 13668-13676 (2000), which is incorporated herein by reference.

Humanization of a non-human anti-IP-10 antibody can also be achieved by simultaneous optimization of framework and CDR residues, which permits the rapid identification of co-operatively interacting framework and CDR residues, as described in Wu et al., J. Mol. Biol. 294(I): 151-162 (1999), which is incorporated herein by reference. Briefly, a combinatorial library that examines a number of potentially important framework positions is expressed concomitantly with focused CDR libraries consisting of variants containing random single amino acid mutations in the third CDR of the heavy and light chains. By this method, multiple Fab variants containing as few as one non-human framework residue and displaying up to approximately 500-fold higher affinity than the initial chimeric Fab can be identified. Screening of combinatorial framework-CDR libraries permits identification of monoclonal antibodies with structures optimized for function, including instances in which the antigen induces conformational changes in the monoclonal antibody. The enhanced humanized variants contain fewer non-human framework residues than antibodies humanized by sequential in vitro humanization and affinity maturation strategies known in the art.

It is further contemplated that an anti-IP-10 antibody of the invention can be a fully human antibody or a primatized antibody. Fully human antibodies can be generated by methods known in the art that involve immunizing a transgenic non-human animal with the desired antigen. The Medarex HuMAb mice and the Kirin Transchromosomic mice are useful for preparing fully human antibodies. The transgenic non-human animal can be modified such that it fails to produce endogenous antibodies, but instead produces B-cells which secrete fully human immunoglobulins. The antibodies produced can be obtained from the animal directly or from immortalized B-cells derived from the transgenic non-human animal.

As used herein a “humanized antibody” is an antibody that contains up to 100% human protein sequences. Humanized antibodies may be derived from other organisms, such as another mammal or vertebrate, but one or more specific amino acids of the antibody are changed, by any technique known in the art, so as to conform more closely to an antibody that would typically be generated in a human or primate. Humanized antibodies may also be generated from rodents, such as transgenic mice carrying a suite of human immunological genes. Humanized antibodies are anticipated to result in a favorable safety profile which are also likely to be eliminated less rapidly from the human body, potentially reducing dosage frequency and amount.

Alternatively, the genes encoding the immunoglobulins with human variable regions can be recovered and expressed to obtain the antibodies directly or modified to obtain analogs of antibodies such as, for example, single chain Fᵥ molecules. Thus, it is contemplated to produce an anti-IP-10 antibody of the invention that is a fully human immunoglobulin to the IP-10 antigen or to produce an analog of the immunoglobulin by a process that includes immunizing a non-human animal with antigen under conditions that stimulate an immune response.

The non-human animal that produces a human antibody of the invention can be modified to be substantially incapable of producing endogenous heavy or light immunoglobulin chain, but capable of producing immunoglobulins with both human variable and constant regions. In the resulting immune response, the animal produces B cells which secrete immunoglobulins that are fully human and specific for the antigen. The human immunoglobulin of desired specificity can be directly recovered from the animal, for example, from the serum, or primary B cells can be obtained from the animal and immortalized. The immortalized B cells can be used directly as the source of human antibodies or, alternatively, the genes encoding the antibodies can be prepared from the immortalized B cells or from primary B cells of the blood or lymphoid tissue, for example, spleen, tonsils, lymph nodes, bone marrow, of the immunized animal and expressed in recombinant hosts, with or without modifications, to produce the immunoglobulin or its analogs. In addition, the genes encoding the repertoire of immunoglobulins produced by the immunized animal can be used to generate a library of immunoglobulins to permit screening for those variable regions which provide the desired affinity. Clones from the library which have the desired characteristics can then be used as a source of nucleotide sequences encoding the desired variable regions for further manipulation to generate human antibodies or analogs with these characteristics using standard recombinant techniques.

Various techniques for preparing human antibodies using transgenic non-human animals, for example, transgenic mice, are well known in the art and described, for example, in Fishwild et al., Nature Biotechnology 14:845-851 (1996); Heijnen et al., Journal of Clinical Investigation 97: 331-338 (1996); Lonberg et al. Nature 368: 856-859 (1994); Morrison, Nature 368: 812-813 (1994); Neuberger, Nature Biotechnologv 14: 826 (1996); Chadd and Chamow, Curr. Opin. Biotechnol. 12(2):188-94 (2001); Russel et al., Infection and Immunity 1820-1826 (2000); Gallo et al., European Journal of Immunology 30:534-540 (2000); Davis et al., Cancer Metastasis Rev. 18.4):421-5 (1999); Green, Journal of Immunological Methods 231:11-23 (1999); Yang et al., Journal of Leukocyte Biology 66:401-410 (1999); Jakobovits, Advanced Drug Delivery Reviews 31:33-42 (1998); Green and Jakobovits, J. Exp. Med. 188(3):483-495 (1998); Jakobovits, Exp. Opin. Invest. Drugs 7(4):607-614 (1998); Mendez et al., Nature Genetics 15:146-156 (1997); Jakobovits, Weir's Handbook of Experimental Immunology, The Integrated Immune System, Vol. IV: 194 194.7 (1996), each of which is incorporated herein by reference. Furthermore, various techniques known in the art for preparation of a human antibody are described in U.S. Pat. Nos. 6,162,963; 6,150,584; 6,114,598; 6,111,166; 6,096,311 and 6,075,181, each of which is incorporated herein by reference.

Another highly efficient means for generating recombinant antibodies is by primatization as described by Newman, Biotechnology 10:1455-1460 (1992), which is incorporated herein by reference. More particularly, primatized antibodies can be generated, which antibodies contain monkey variable domains and human constant sequences.

Methods for primatization are known in the art and described in U.S. Pat. No. 6,113,898, which is incorporated herein by reference. Antibodies can be primatized such that they are not antigenically rejected upon human administration. Primatization relies on immunization of primates, for example, cynomolgus monkeys (Macaca fascicularis), with human antigens or receptors and can be used to generate high affinity monoclonal antibodies directed to human cell surface antigens.

Antibodies generated by primatization have previously been reported to display human effector function, have reduced immunogenicity, and long serum half-life and are thus useful as therapeutic agents of the present invention. The technology relies on the fact that despite the fact that cynomolgus monkeys are phylogenetically similar to humans, they still recognize many human proteins as foreign and therefore mount an immune response.

Moreover, because the cynomolgus monkeys are phylogenetically close to humans, the antibodies generated in these monkeys have been discovered to have a high degree of amino acid homology to those produced in humans. Indeed, after sequencing macaque immunoglobulin light and heavy chain variable region genes, it was found that the sequence of each gene family was 85-98% homologous to its human counterpart (Newman et al, supra, 1992). The first antibody generated by primatization, an anti-CD4 antibody, was 91-92% homologous to the consensus sequence of human immunoglobulin framework regions (Newman et al. supra, 1992). Methods known in the art for generation of a primatized antibody and useful for preparing an anti-IP-10 antibody of the invention further are described by Newman et al., Clinical Immunology 98(2):164-74(2001); and Reddy et al., J. Immunol. 164(4):1925-33 (2000), both of which are incorporated herein.

As described above, anti-IP-10 antibodies of the invention include, for example, polyclonal antibodies, monoclonal antibodies, humanized antibodies, as well as recombinant versions and functional fragments thereof. Recombinant versions of these antibodies include a wide variety of constructions ranging from simple expression and co-assembly of encoding heavy and light chain cDNAs to specialty constructs termed designer antibodies. Recombinant methodologies, combined with the extensive characterization of polypeptides within the immunoglobulin superfamily, and particularly antibodies, provides the ability to design and construct a vast number of different types, styles and specificities of binding molecules derived from immunoglobulin variable and constant region binding domains. Specific examples include chimeric antibodies, where the constant region of one antibody is substituted with that of another antibody, and humanized antibodies, described above, where the complementarity determining regions (CDR) from one antibody are substituted with those from another antibody.

Other recombinant versions of antibody may include, for example, functional antibody variants where the variable region binding domain or functional fragments responsible for maintaining antigen binding is fused to an Fc receptor binding domain from the antibody constant region. Such variants are essentially truncated forms of antibodies that remove regions non-essential for antigen and Fc receptor binding. Truncated variants can have single valency, for example, or alternatively be constructed with multiple valencies depending on the application and need of the user. Additionally, linkers or spacers can be inserted between the antigen and Fc receptor binding domains to optimize binding activity as well as contain additional functional domains fused or attached to effect biological functions other than IP-10 neutralization. Those skilled in the art will know how to construct recombinant antibodies specific for IP-10 in light of the art knowledge regarding antibody engineering and given the guidance and teachings herein. A description of recombinant antibodies, functional fragments and variants and antibody-like molecules can be found, for example, in “Antibody Engineering,” 2nd Edition, (Carl A. K. Borrebaeck, Ed.) Oxford University Press, New York,(1995).

Additional functional variants of antibodies that can be used as antibody therapeutic agents include antibody-like molecules other than antigen binding-Fc receptor binding domain fusions. For example, antibodies, functional fragments and fusions thereof containing a Fc receptor binding domain can be produced to be bispecific in that one variable region binding domain exhibits binding activity for one antigen and the other variable region binding domain exhibits binding activity for a second antigen. Such bispecific antibodies can be advantageous in the methods of the invention because a single bispecific antibody will contain two different target antigen binding species. Therefore, a single molecular entity can be administered to achieve neutralization of, for example, both IP-10 and another chemokine. Such antibodies are also considered “anti-IP-10 antibodies” in this disclosure.

An “anti-IP-10 antibody” can also be an immunoadhesion or bispecific immunoadhesion. Immunoadhesions are antibody-like molecules that combine the binding domain of a non-antibody polypeptide with the effector functions of an antibody of an antibody constant domain. The binding domain of the non-antibody polypeptide can be, for example, a ligand or a cell surface receptor having ligand binding activity. Immunoadhesions for use as anti-IP-10 antibodies can contain at least the Fc receptor binding effector functions of the antibody constant domain. Specific examples of ligands and cell surface receptors that can be used for the antigen binding domain of an immunoadhesion therapeutic agent include, for example, a T cell or NK cell receptor such as the CXCR3 receptor that recognizes IP-10.

Other ligands and ligand receptors known in the art can similarly be used for the antigen binding domain of an immunoadhesion anti-IP-10 antibody. In addition, multivalent and multispecific immunoadhesions can be constructed for use as anti-IP-10 antibodies. The construction of bispecific antibodies, immunoadhesions, bispecific immunoadhesions and other heteromultimeric polypeptides which could be used according to this invention as IP-10 specific antibodies is the subject matter of, for example, U.S. Pat. Nos. 5,807,706 and 5,428,130, which are incorporated herein by reference.

Antibody therapeutic agents which have been approved for marketing include Orthoclone OKT3® (Johnson & Johnson), ReoPro® (Lilly), Rituxan® (Genentech), Simulect® (Novartis), Remicade® (Johnson & Johnson), Zenapax® (Roche), Synagis® (Medimmune), Herceptin® (Genentech), Mylotarg® (American Home Products), Campath® (Millennium), Zevalin® (IDEC Pharmaceuticals and Schering AG), and Humira® (Abbott Laboratories). These agents use various antibody formats including murine, chimeric, CDR-grafted, radiolabeled and phage display. Current strategies for antibody development by leading companies such as Abgenix, Inc. (Freemont, Calif.), Protein Design Labs, Inc. (Fremont Calif.), and Medarex, Inc. (Princeton N.J.) emphasize the preference for humanized antibody formats.

Combination Therapies Using Anti-IP-10 Antibody for the Treatment of Multiple Sclerosis

A wide variety of therapeutic compounds are currently in use or in development for the treatment of Multiple Sclerosis. The invention pertains to the use of anti-IP-10 antibody in combination with at least one of these therapeutic compounds.

TABLE 1
Therapeutic compounds useful in combination therapy

| Compound | Trade Name | Vendor | Target | Dosing |
|---|---|---|---|---|
| Chronic | | | | |
| progression | | | | |
| Beta-interferon (1a) | Rebif ® | Serono | Immunomodulator | 44 micrograms |
| | | | | (mcg) three |
| | | | | times per week |
| | | | | by |
| | | | | subcutaneous |
| | | | | injection |
| Beta-interferon (1a) | Avonex ® | Biogen | Immunomodulator | 30 mcg injected |
| | | | | intramuscularly |
| | | | | once a week |
| Beta-interferon (1b) | Betaseron ® | Berlex | Immunomodulator | 0.25 mg injected |
| | | | | subcutaneously |
| | | | | every other day |
| Glatiramer acetate | Copaxone ® | Teva/ | Immunomodulator | 20 mg/day |
| | | Aventis | | injected |
| | | | | subcutaneously |
| Mitoxantrone | Novantrone ® | Amgen/ | Immunosuppressant- | Once every |
| | | Serono | | three months |
| Exacerbation | | | | |
| Methylprednisolone | Solu-Medrol | | Anti-inflammatory | 200 mg daily for |
| | | | glucocorticoid | 1 week followed |
| | | | | by 80 mg every |
| | | | | other day for |
| | | | | one month |
| Dexamethasone | Decadron | | Anti-inflammatory | Dosage varies |
| | | | synthetic | from 0.75 to 9 mg |
| | | | adrenocortical | a day, |
| | | | steroid | although it may |
| | | | | be higher or |
| | | | | lower in specific |
| | | | | conditions. |
| Betamethasone | | | Anti-inflammatory | Dosage varies |
| | | | synthetic | from 0.75 to 9 mg |
| | | | glucocorticoid | a day, |
| | | | | although it may |
| | | | | be higher or |
| | | | | lower in specific |
| | | | | conditions. |
| Symptom | | | | |
| Management | | | | |
| 4-amino-3-(4- | Baclofen | | muscle relaxant and | |
| chlorophenyl)- | | | antispastic. | |
| butanoic acid. | | | | |
| 4-aminopyridine (4- | | | potassium channel | |
| AP) and 3,4- | | | blocker | |
| diaminopyridine | | | | |
| (3,4-DAP) | | | | |

The compounds of Table 1 may be employed in combination with humanized anti-IP-10 antibody according to the methods of this invention.

TABLE 2
Therapeutic compounds useful in
combination therapy for reducing MS symptoms

| Compound | Target | Dosing | Sponsor |
|---|---|---|---|
| Natalizumab | anti-VLA-4 humanized | IV once a | Biogen/Elan |
| (Antegren) | antibody (inhibitor of alpha | month | |
| | 4 integrin (alpha-4-beta-1 | | |
| | (VLA-4) and alpha-4-beta- | | |
| | 7) | | |
| Teriflunomide | Dihydroorotate | Oral | Aventis |
| (HMR 1726) | dehydrogenase inhibitor; | | |
| | protein tyrosine kinase | | |
| | inhibitor; | | |
| | immunosuppressant | | |
| | (inhibits de novo pyrimidine | | |
| | synthesis, anti-inflammatory | | |
| | action) | | |
| Mesopram | PDE-IV inhibitor | Oral | Schering AG |
| (ZK 117137) | | | |
| Perfenidone | FGF, TGF-beta and PDGF | | Schering AG |
| | Cytokine antagonist | | |
| Tiplimotide | Analog of myelin basic | | Neurocrine, |
| | protein fragment | | Inc. |
| MBP-8298 | Analog of myelin basic | | Univ. of |
| | protein fragment | | Alberta |
| fampridine | Potassium channel blocker | | Elan/ |
| (EL-970; 4- | | | Acorda |
| aminopyridine) | | | |
| Methotrexate | Synthetic | | |
| | immunosuppressive | | |
| PEG-Rebif | Pegylated-Rebif ® | | Inhale |
| | | | Therapeutics |
| BX-471 (or | CCR1 chemokine | | Schering AG |
| CCR-1) (ZK | antagonist | | |
| 811752) | | | |
| EMZ-701 & | unknown | | Transition |
| IFN-beta | | | Therapeutics |
| ShK | T-Cell ion channel blocker | | |
| | peptide | | |
| GGF-2 | Neuregulin growth factor | | Acorda |
| NRG-2 | unknown | | Acorda |
| M1 mAbs | unknown | | Acorda/ |
| | | | Mayo |

Anti-IP-10 antibody may also be used in combination with therapeutic compounds in Table 2, according to the methods of the invention.

Preferred Combination Therapies

While not intending to be bound to any specific mechanism of action, it is believed that anti-IP-10 antibody treats chronic progression of MS and reduces the severity of MS symptoms by removing or reducing the chemokine IP-10 from the vicinity of neurons and inflamed tissue surrounding the neurons, thereby preventing or reducing the accumulation of T-cells, which are believed to be responsible for chronic and persistent damage to neurons and surrounding tissues.

The most effective combination therapies are likely those that employ therapeutic mechanisms of action for Multiple Sclerosis management that are complementary to the mechanism of action of anti-IP-10 antibodies. Thus, immunomodulators, such as, for example, beta interferons and glatiramer acetate, as well as anti-VLA-4 antibody in combination with and anti-IP-10 antibody provide complementary and synergistic effects. On the other hand, immunosuppressors, such as mitoxantrone, which generally deplete T-cell populations are less suitable for combination therapy with anti-IP-10 antibody and will not provide cumulative benefits.

The success of the combination therapy of the present invention depends upon proper dosing and administration of humanized anti-IP-10 antibody with the appropriate combination therapy candidate. A variety of proposed dosage regimens are provided below. It is within the skill of the art to select an appropriate dosing regimen that will optimize safety and efficacy. The humanized anti-IP-10 antibody will be administered in a dose sufficient to enhance the effectiveness of the combination therapy candidate, which together will treat chronic progression of MS or reduce MS symptoms in an individual. An amount of anti-IP-10 antibody adequate to accomplish this is defined as a “therapeutically effective” dose. Therapeutically effective doses of anti-IP-10 antibody will vary depending upon many different factors, including means of administration, target site, physiological state of the patient, judgment of the physician, and interaction with the other combination therapy candidates. Similarly, therapeutically effective doses of combination therapy candidate will vary depending upon many factors, including means of administration, physiological state of the patient, judgment of the physician and interaction with humanized anti-IP-10 antibodies. As used herein, “combination therapy candidates” refers to therapeutic compounds useful for treating chronic progression of MS or reducing the severity of MS symptoms. Examples of combination therapy candidates useful in the present invention are listed in Tables 1 and 2. At least one combination therapy candidate is used together with the humanized anti-IP-10 antibody of the present invention to form the combination therapy of the present invention.

In general, therapeutically effective doses of anti-IP-10 antibody range from about 0.0001 to about 100 milligrams of antibody per kilogram body weight of individual to whom it is administered (mg/kg), more usually from about 1 to about 15 mg/kg, preferably from about 5 to about 10 mg/kg and most preferably about 8 mg/kg. Accordingly, given an average human weighing 70 kg, a therapeutically effective dose of anti-IP-10 antibody ranges from about 0.0070 mg to about 7000 mg, more usually from about 70 mg to about 1050 mg, preferably from about 350 mg to about 700 mg, and most preferably about 560 mg per dose.

Pharmaceutical compositions of the present invention comprising anti-IP-10 antibody will include a pharmaceutically acceptable carrier for the antibody. The pharmaceutical compositions of the present invention will be administered by parenteral, oral, intravenous, subcutaneous, intrathecal, intramuscular or other local delivery technique. Preferred techniques will give the anti-IP-10 antibody suitable access to the central nervous system and spinal cord where IP-10 accumulates and also provide the combination therapy compound the opportunity to exert its effect on the individual. In one embodiment, the humanized anti-IP-10 antibody of the present invention may be formulated in an aqueous carrier according to techniques well known to those of skill in the art, which techniques may include formulation in, for example, water, buffered water, saline, glycine, hyaluronic acid and the like. Such aqueous compositions may be sterilized by conventional methods known to those of skill in the art or, alternatively, may be sterile filtered. Additionally, the aqueous solutions of the present invention may be packaged for use as is or may be lyophilized to be reconstituted with sterile solution prior to administration. It is to be understood that aqueous solutions may contain pharmaceutically acceptable excipients, such as, for example, buffering agents, tonicity adjusting agents, wetting agents and the like.

In another embodiment, the present invention contemplates the humanized anti-IP-10 antibodies and combination therapy candidates packaged in kits. The kits of the present invention may comprise one or more containers filled with aqueous solutions of humanized anti-IP-10 antibodies separately or together with a particular combination therapy candidate. For example, a kit of the present invention may comprise one vial of humanized anti-IP-10 antibody and a separate vial comprising a therapeutically effective amount of an immunomodulator. Alternatively, the kit may comprise a single vial containing therapeutically effective amounts of humanized anti-IP-10 antibodies together with a therapeutically effective amount of a combination therapy candidate, such as, for example, an immunomodulator. A kit of the present invention may optionally include means for administering the combination therapy to an individual in need thereof, as well as instructions for proper dosing use.

Those skilled in the art are aware that preferred dosing may be daily, every 2, 3, 4, 5 or 6 days, weekly, every 2, 3, 4, 5, 6, 7, or 8 weeks, or monthly, or every 2, 3, 4, 5, or 6 months. Because of the long circulating half-life of humanized anti-IP-10 antibodies, preferred dosing may be at least 2 days, at least 5 days, at least 1 week, at least 2 weeks, at least 3 weeks, at least 4 weeks, at least 6 weeks or at least 8 weeks. Moreover, intravenous dosing of about once per month may be preferred to maintain sufficiently high levels of humanized anti-IP-10 antibody to treat chronic progression of MS. More regular dosing of humanized anti-IP-10 antibody to treat chronic progression of MS, if necessary, will preferably employ an intramuscular administration.

Treatment with chronic progression combination therapy likely will be most enhanced by approximately monthly dosing of humanized anti-IP-10 antibody, although other dosing schedules are possible. Preferred combinations for treating chronic progression, which examples are not intended to limit the scope of options set out herein, include:

| Avonex ® | Weekly/intramuscular/30 mcg |
|---|---|
| Anti-IP-10 antibody | Monthly/intravenous/5-10 mg/kg |
| Avonex ® | Weekly/intramuscular/30 mcg |
| Copaxone ® | Daily/subcutaneous/20 mg |
| Anti-IP-10 Antibody | Monthly/intravenous/5-10 mg/kg |
| Interferon | As per Avonex ®, Betaseron ®, Rebif ® |
| Anti-VLA-4 Antibody | Monthly/intravenous/0.1-100 mg/kg |
| Anti-IP-10 Antibody | Monthly/intravenous/5-10 mg/kg |
| Antibodies administered within 24 hours of each other. | |
| Interferon | As per Avonex ®, Betaseron ®, Rebif ® |
| Anti-VLA-4 Antibody | Monthly/intravenous/0.1-100 mg/kg |
| Anti-IP-10 Antibody | Monthly/intravenous/5-10 mg/kg |

Antibodies administered sequentially in alternating 2 week
intervals.

Treatment with combination therapy likely for reducing the severity of MS symptoms will be most enhanced by approximately monthly dosing of humanized anti-IP-10 antibody, although other dosing schedules are possible. Preferred combinations for reducing the severity of MS symptoms, which examples are not intended to limit the scope of options set out herein, include:

| | |
|---|---|
| Teriflunomide | Weekly/oral |
| Anti-IP-10 antibody | Monthly/intravenous/5-10 mg/kg |
| Mesopram | Weekly/oral |
| Anti-IP-10 antibody | Monthly/intravenous/5-10 mg/kg |
| PEG-Rebif | Weekly |
| Anti-IP-10 antibody | Monthly/intravenous/5-10 mg/kg |

The following examples are offered to illustrate various aspects of the invention and are not to be construed as to limit the invention in any way.

### EXAMPLES

### Example 1

### Generation of Humanized Anti-IP-10 Antibody

Humanized anti-IP-10 antibodies may be generated by selecting an appropriate immunogen comprising a nucleotide sequence of IP-10 and injecting that immunogen into the HuMAb-Mouse® (Medarex, Inc.) to subsequently generate monoclonal antibodies directed against IP-10 protein.

### Example 2

### MOG 35-55 Peptide Induction of EAE

Confirmation of the beneficial effects of combination therapies using anti-IP-10 antibody for the treatment of MS may be obtained through human clinical trials and/or the use of animal models which parallel the disease process of MS in humans. A preferred animal model of human Multiple Sclerosis is the induction of experimental autoimmune encephalomyelitis (EAE) in mice and rodents. EAE is induced in strains of lab animals that are susceptible to the disease by injecting myelin or myelin specific proteins in combination with an immune-stimulating adjuvant. Adoptive induction of EAE with proteolipid protein (PLP), is detailed in Current Protocols in Immunology (Chapter 15) “Adoptive induction of EAE with PLP or MBP specific lymphocytes” Contributors: Stephen D. Miller and William J. Karpus (Edited by: John E. Coligan, Ada M. Kruisbeek, David H. Margulies, Ethan M. Shevach, Warren Strober; John Wiley & Sons). For the induction of EAE with myelin oligodendrocyte glycoprotein (MOG) an excellent reference is Mendel I., Kerlero de Rosbo N., and A. Ben-Nun. 1995. A myelin oligodendrocyte glycoprotein peptide induces typical chronic experimental autoimmune encephalomyelitis in H-2b mice: fine specificity and T cell receptor V beta expression of encephalitogenic T cells. European Journal of Immunology 25: 1951 (and subsequent citations therein).

MOG 35-55 (GPGYPIRALVGDEAELPCRIS) peptide is prepared by reconstitution in 30% DMF. Complete Freund's adjuvant (CFA) is prepared by adding 40 mg of M. Tuberculosis (Difco cat#DF3114-33-8) to incomplete Freund's adjuvant (IFA) (Sigma cat#F5506). A 1:1 mixture of peptide and CFA emulsion is prepared and 200 μl is injected sub-cutaneously (s.c) on the flank of the mouse (C57BL/6) 5-7 weeks of age (Jackson Labs cat#000664). Immediately following immunization, 400 ng of pertussis toxin (Sigma cat#P7028) is injected intravenously in 500 μl of PBS. Forty-eight hours after the first immunization, a booster injection (intravenous) of 400 ng pertussis toxin is performed. Seven days following the first immunization with MOG 35-55, an identical immunization (sbucutaneous) of MOG 33-55 emulsified in CFA is administered on the opposite flank of the mouse. The mice are monitored for disease development (typically 11-12 days post-immunization).

### Example 3

### Adoptive Transfer of EAE with PLP

CFA is prepared by mixing 10 ml of IFA with 40 mg of M. Tuberculosis. An emulsion of PLP 139-151 (HSLGKWLGHPDKF) and CFA is made by mixing 1 ml of 2 mg/ml PLP with 1 ml of the CFA. The backs of Female SJL mice 5-8 weeks old (Jackson Labs cat#000686) are shaved with small animal clippers. A 100 μl emulsion is injected (subcutaneous) in the shaved backs of mice, distributing over three sites (33 μl /site): one along the midline of back between the shoulders, and two on either side of the midline on the lower back. Seven to 10 days following immunization, the mice are sacrificed and the draining lymph nodes (inguinal, brachial and axillary) are removed and placed in Hanks balance salt solution. A single cell suspension is generated. PLP-primed cells are prepared at a concentration of 6×10⁶ cells/ml in complete DMEM 5% FCS containing 50 μg/ml of PLP. The cell suspension is Incubated for 72 hours in a 37° C. 7.5% CO₂ tissue culture incubator. PLP-activated lymphocytes are harvested by centrifuging in 50 ml conical tube for 15 minutes at 300×g. The harvested lymphocytes are washed and centrifuged as before with HBSS. Cells are resuspended in HBSS and 1×10⁷ viable cells in 0.5 ml HBSS are injected intraperitoneally into normal recipient SJL mice. The mice are monitored for disease development (typically 7-8 days post-transfer).

### Example 4

### Combination Therapy with Type-1 Interferon and Anti-IP-10 Antibodies

Mice from both MOG 35-55 and PLP 139-151-induced EAE models will be treated every other day individually with either saline, IFN-α/B (subcutaneous) (R&D cat#12100-1, 12400-1), or anti-IP-10 (intraperitoneal) (Ability Biomedical) after the appearance of clinical symptoms for 22 days. Preliminary experiments will be performed to determine the optimal effective dose of each type of treatment. For combination treatments, suboptimal doses of type-I interferons and anti-IP-10 treatments will be administered subcutaneously and intraperitoneally respectively after the appearance of clinical symptoms in order to document any additive effects of the combination treatment.

Mice will be monitored for improvement in clinical symptoms throughout the course of treatment, and sacrificed at day 30 post-immunization when spinal cords will be removed and examined for demyelination, and inflammatory cell infiltration. Clinical scores will be assessed. In brief, animals will be graded according clinical severity as follows: 0, no abnormality; 1, limp tail; 2 limp tail and hind-limb weakness (waddling gait); 3, partial hind-limb paralysis; 4 complete hind-limb paralysis; 5, death according to the protocol described at Fife et al. (2001), CXCL10 control of encephalitogenic CD4+ T cell accumulation in the central nervous system during experimental autoimmune encephalomyelitis. J. Immunol. 166: 7617-7624, which is incorporated by reference herein.

Demyelination will be determined by staining with Luxol fast blue (LFB) on paraffin embedded spinal cord sections. Histological scores will be determined as using the following scale: 0, no demyelination: 1, mild inflammation accompanied by loss of myelin integrity; 2, moderate inflammation with increasing myelin damage; 3, numerous inflammatory lesions accompanied by significant increase in myelin stripping; and 4, intense areas of inflammation accompanied by numerous phagocytic cells engulfing myelin debris according to the protocol described in Liu M. T., Keirstead H. S., Lane T. E. (2001) Neutralization of the chemokine CXCL10 reduces inflammatory cell invasion and demyelination and improves neurological function in a viral model of Multiple Sclerosis. J. Immunol. 167:4091-7, which is incorporated by reference herein.

Inflammatory cell infiltration will be determined by staining with hematoxylin and eosin on paraffin embedded spinal cord sections. Inflammation will be scored as follows: 0, no mononuclear cell infiltration; 1, 1-5 perivascular lesions per section with parenchymal infiltration; 2, 5-10 perivascular lesions per section with parenchymal infiltration; and 3>10 perivascular lesions per section with extensive parenchymal infiltration, as per Fife et al. (2001), infra.

The present invention may be embodied in other specific forms without departing from the teachings or essential characteristics of the invention. The described embodiments are therefore to be considered in all respects as illustrative and not restrictive, the scope of the invention being indicated by the appended claims rather than the foregoing description, and all changes that come within the meaning and range of equivalency of the claims are to be embraced within.

1. A method for treating chronic progression of Multiple Sclerosis in an individual, the method comprising administering to the individual a therapeutically effective amount of a humanized anti-IP-10 antibody in combination with a therapeutically effective amount of a compound selected from the group consisting of beta-interferon 1a, beta-interferon 1b, humanized anti-VLA-4-antibody, and glatiramer acetate.

2. The method of claim 1, wherein the anti-IP-10 antibody is a humanized antibody.

3. The method of claim 1, wherein the anti-IP-10 antibody is a human antibody.

4. The method of claim 1, wherein the chronic progression treated is the remitting phase of relapsing-remitting Multiple Sclerosis.

5. The method of claim 1, wherein the chronic progression treated is the secondary progressive form of Multiple Sclerosis.

6. The method of claim 1, wherein the chronic progression treated is the primary progressive form of Multiple Sclerosis.

7. The method of claim 1, wherein the therapeutically effective amount of humanized anti-IP-10 antibody is administered in a dose ranging from about 1 mg/kg to about 15 mg/kg.

8. The method of claim 1, wherein the therapeutically effective amount of humanized anti-IP-10 antibody is administered in a dose ranging from about 5 mg/kg to about 10 mg/kg.

9. The method of claim 1, wherein the therapeutically effective amount of humanized anti-IP-10 antibody is administered in a dose of about 8 mg/kg.

10. The method of claim 1, wherein the therapeutically effective amount of humanized anti-IP-10 antibody is administered about once every 3 weeks.

11. The method of claim 1, wherein the therapeutically effective amount of humanized anti-IP-10 antibody is administered about once every month.

12. A method for reducing the severity of Multiple Sclerosis symptoms in an individual in need thereof, the method comprising administering to the individual a therapeutically effective amount of a humanized anti-IP-10 antibody in combination with a therapeutically effective amount of a compound selected from the group consisting of 4-amino-3-(4-chlorophenyl)-butanoic acid, 4-aminopyridine, 3,4-diaminopyridine, antegren, HMR 1726, ZK 117137, perfenidone, tiplimotide, MBP-8298, fampridine, methotrexate, PEG-Rebif, BX-471, EMZ-701, Shk, GGF-2, NRG-2, and M1 mAbs.

13. The method of claim 12, wherein the anti-IP-10 antibody is a humanized antibody.

14. The method of claim 12, wherein the anti-IP-10 antibody is a human antibody.

15. The method of claim 12, wherein the chronic progression treated is the remitting phase of relapsing-remitting Multiple Sclerosis.

16. The method of claim 12, wherein the chronic progression treated is the secondary progressive form of Multiple Sclerosis.

17. The method of claim 12, wherein the chronic progression treated is the primary progressive form of Multiple Sclerosis.

18. The method of claim 12, wherein the therapeutically effective amount of humanized anti-IP-10 antibody is administered in a dose ranging from about 1 mg/kg to about 15 mg/kg.

19. The method of claim 12, wherein the therapeutically effective amount of humanized anti-IP-10 antibody is administered in a dose ranging from about 5 mg/kg to about 10 mg/kg.

20. The method of claim 12, wherein the therapeutically effective amount of humanized anti-IP-10 antibody is administered in a dose of about 8 mg/kg.

21. The method of claim 12, wherein the therapeutically effective amount of humanized anti-IP-10 antibody is administered about once every 3 weeks.

22. The method of claim 12, wherein the therapeutically effective amount of humanized anti-IP-10 antibody is administered about once every month.

23. A kit useful for treating chronic progression of Multiple Sclerosis in an individual, the kit comprising:
a therapeutically effective amount of a humanized anti-IP-10 antibody;
a therapeutically effective amount of a compound selected from the group consisting of beta-interferon 1a, beta-interferon 1b, humanized anti-VLA-4-antibody, and glatiramer acetate;
means for administering the antibody and the compound in combination with each other; and
instructions for determining proper dosing and administration of the combination to an individual.

24. A kit useful for reducing the severity of Multiple Sclerosis symptoms in an individual in need thereof, the kit comprising:
a therapeutically effective amount of a humanized anti-IP-10 antibody;
a therapeutically effective amount of a compound selected from the group consisting of 4-amino-3-(4-chlorophenyl)-butanoic acid, 4-aminopyridine, 3,4-diaminopyridine, antegren, HMR 1726, ZK 117137, perfenidone, tiplimotide, MBP-8298, fampridine, methotrexate, PEG-Rebif, BX471, EMZ-701, Shk, GGF-2, NRG-2, and M1 mAbs;
means for administering the antibody and the compound in combination with each other; and
instructions for determining proper dosing and administration of the combination to an individual.

25. A method for preventing chronic progression of Multiple Sclerosis in an individual, the method comprising:
administering to the individual a therapeutically effective amount of a humanized anti-IP-10 antibody in combination with a therapeutically effective amount of a compound selected from the group consisting of beta-interferon 1a, beta-interferon 1b, humanized anti-VLA-4-antibody, and glatiramer acetate; and
determining whether chronic progression of Multiple Sclerosis has been prevented in the individual.

26. The method of claim 25, wherein the anti-IP-10 antibody is a humanized antibody.

27. The method of claim 25, wherein the anti-IP-10 antibody is a human antibody.

28. The method of claim 25, wherein the chronic progression treated is the remitting phase of relapsing-remitting Multiple Sclerosis.

29. The method of claim 25, wherein the chronic progression treated is the secondary progressive form of Multiple Sclerosis.

30. The method of claim 25, wherein the chronic progression treated is the primary progressive form of Multiple Sclerosis.

31. The method of claim 25, wherein the step of determining whether chronic progression of Multiple Sclerosis has been prevented comprises:
subjecting the individual to a clinical or biochemical test selected from the group consisting of relapse rate, vision loss, sensory loss, gait, bladder dysfunction, depression, cognitive impairment, EDSS, MSFC, MTR, NAA/Cr, myelin level, integrity of the blood-brain barrier, perivascular infiltration of mononuclear cells, immunologic abnormalities, gliotic scar formation, astrocyte proliferation, mettaloproteinase production and impaired conduction velocity.

32. A method for reducing the severity of Multiple Sclerosis symptoms in an individual in need thereof, the method comprising:
administering to the individual a therapeutically effective amount of a humanized anti-IP-10 antibody in combination with a therapeutically effective amount of a compound selected from the group consisting of 4-amino-3-(4-chlorophenyl)-butanoic acid, 4-aminopyridine, 3,4-diaminopyridine, antegren, HMR 1726, ZK 117137, perfenidone, tiplimotide, MBP-8298, fampridine, methotrexate, PEG-Rebif, BX-471, EMZ-701, Shk, GGF-2, NRG-2, and M1 mAbs; and
determining whether the severity of Multiple Sclerosis symptoms has been reduced in the individual.

33. The method of claim 32, wherein the anti-IP-10 antibody is a humanized antibody.

34. The method of claim 32, wherein the anti-IP-10 antibody is a human antibody.

35. The method of claim 32, wherein the chronic progression treated is the remitting phase of relapsing-remitting Multiple Sclerosis.

36. The method of claim 32, wherein the chronic progression treated is the secondary progressive form of Multiple Sclerosis.

37. The method of claim 32, wherein the chronic progression treated is the primary progressive form of Multiple Sclerosis.

38. The method of claim 32, wherein the step of determining whether the severity of Multiple Sclerosis symptoms has been reduced comprises:
subjecting the individual to a test that measures an arrest, decrease, or reversal in signs, physiological indicators, biochemical markers, or metabolic indicators associated with MS, wherein the test is selected from the group consisting of neurological impairment, neuroloinflammation, demyelination of nerve fibers, remyelination of nerve fibers, CT scans, MRI scans for enhancing lesions, MRI scans for T1 black holes, MRI scans for T2 lesion load, level of myelin, and level of gamma globulin.
